(19) United States
(12) Patent Application Publication
(10) Pub. No.: US 2026/0199701 A1
(43) Pub. Date: Jul. 16, 2026

(54) THERAPEUTIC CONTROLLER AND A DEVICE

(71) Applicant: Shenzhen Kaiyan Medical Equipment Co., Ltd., Shenzhen (CN)

(72) Inventors: Alain Dijkstra, Amstelveen (NL); Li Xiang, Shenzhen (CN); Fan Lin, Shenzhen (CN); Li Quan, Shenzhen (CN)

(73) Assignee: Shenzhen Kaiyan Medical Equipment Co., Ltd., Shenzhen (CN)

(21) Appl. No.: 19/354,868

(22) Filed: Oct. 10, 2025

(30) Foreign Application Priority Data
Oct. 12, 2024 (CN) 202422476612X
Oct. 12, 2024 (CN) 2024224730221
Oct. 12, 2024 (CN) 2024224736247

(51) Int. Cl. A61N 5/06

(52) U.S. Cl. CPC A61N 5/0622; A61N 2005/0626; A61N 2005/0647

(57) ABSTRACT

The present invention relates to a multifunctional therapeutic system comprising a handheld controller, a therapeutic device, and a base for secure placement and operation. The handheld controller includes a housing accommodating a rechargeable battery, control board, button module, display screen, and a phototherapy assembly with lamp beads and concentrator lenses, enabling localized body treatment. The therapeutic device may be a light therapy mask, cap, pad, or other treatment module, comprising inner and outer shells, light panels, and positioning structures to ensure comfort, precise fit, and uniform illumination. The base includes slots and receiving cavities configured to hold the controller and therapeutic device, allowing secure mounting, easy insertion, and removal. The system supports independent or simultaneous operation of the controller and therapeutic device, enhancing versatility, usability, and user comfort while delivering effective phototherapy across multiple body regions.

### TECHNICAL FIELD

The present invention relates to the field of beauty care and physiotherapeutic equipment, and more particularly to a multifunctional therapeutic controller. The invention provides a control apparatus that integrates a self-contained therapeutic module within the controller body while also being operable to connect with and control external therapeutic devices such as phototherapy masks, therapeutic pads, and similar treatment modules. The invention thus resides in the technical field of multifunctional therapeutic systems combining phototherapy emission, device control, and modular therapeutic adaptability.

### BACKGROUND

Traditional phototherapy devices can generally be divided into two categories: standalone units and wearable units. Standalone units, such as portable light panels or handheld emitters, usually consist of lamp beads mounted on a circuit board inside a housing. In some cases, these devices employ focusing lenses or condenser structures to improve the concentration of light delivered to the skin. Although such devices are effective in providing localized phototherapy, they lack adaptability, as their fixed design restricts treatment to specific body regions. Users often require multiple devices to address different areas, which increases cost and reduces convenience.

Wearable devices, such as phototherapy masks and therapeutic pads, have been developed to provide more comprehensive coverage for areas like the face and neck. These devices improve skin contact and treatment efficiency, but they are often uncomfortable due to heat build-up during prolonged use. Many existing masks allow unwanted light to enter the eyes, causing discomfort and safety concerns, while others lack adequate ventilation or ergonomic fit. Moreover, these devices are typically limited to facial treatment and, at best, extend to the neck. They rarely address wider body areas, and because they rely on external controllers or power supplies, they lack portability and independence.

Some advancements have introduced modular systems in which a base and detachable controller are connected to wearable masks or pads. These systems improve flexibility by allowing components to be attached or removed as needed. However, in such arrangements, the controller merely acts as a passive power and control interface. It does not itself provide therapeutic treatment, and its usefulness is entirely dependent on external devices. As a result, even though these designs move toward modularity, they fail to offer a controller that is multifunctional in its own right.

The absence of a device that unites both functions, operating as an intelligent control hub for external therapeutic modules while simultaneously serving as a standalone phototherapy tool, represents a clear gap in the market. Users currently face a fragmented landscape in which standalone devices, wearable masks, and modular systems each solve part of the problem, but none provide a comprehensive solution. This creates inconvenience, drives up costs, and limits user flexibility in choosing appropriate treatment options.

The present invention fills this gap by introducing a multifunctional therapeutic controller. Unlike traditional controllers, which can only power and control attached devices, the proposed invention incorporates its own phototherapy module within the housing. This enables the controller itself to provide localized therapeutic treatment, making it functional and valuable even when used independently. At the same time, the controller is equipped with connection interfaces and control circuitry to attach to and manage external devices such as phototherapy masks, pads, or other therapeutic accessories.

Through this dual functionality, the invention achieves significant advantages. It consolidates the roles of multiple devices into one, thereby reducing costs and simplifying the user experience. It enhances portability, as the controller can be carried and used independently, while still retaining the ability to coordinate external therapeutic devices when needed. It improves market value by addressing the shortcomings of earlier solutions, offering both versatility and convenience in a single system. Thus, the multifunctional therapeutic controller provides an integrated and user-centered phototherapy solution that effectively bridges the gap between existing standalone, wearable, and modular systems.

### OBJECTS OF THE INVENTION

Some of the objects of the invention are as follows:

An object of the present invention is to provide a multi-functional therapy system comprising a first therapy device and a therapeutic controller, each configured with stimulation elements capable of delivering distinct therapeutic modalities to different regions of the body.

An object of the present invention is to provide a coordinated dual-device therapy system wherein the first therapy device and the therapeutic controller can operate in a synchronized, sequential, or independent manner to enhance therapeutic effectiveness and convenience.

Another object of the invention is to enable simultaneous or alternating delivery of multiple therapies, such as phototherapy, microcurrent stimulation, vibration, or thermal treatment, for improved physiological and neural stimulation effects.

Another object of the present invention is to provide a wired or wireless interconnection mechanism between the first therapy device and the therapeutic controller, enabling one device (e.g., handheld controller) to operate as a control hub for another device, such as a wearable mask, pad, or cap.

Another object of the invention is to ensure multi-region therapy delivery, wherein the first therapy device and the second stimulation unit integrated in the controller can target separate anatomical zones, such as face and neck, or head and shoulder, concurrently.

A further object of the invention is to provide a therapeutic controller integrated with physiological sensors, such as temperature, impedance, or heart rate sensors, for adaptive control and personalized therapy adjustment based on real-time body feedback.

Another object of the invention is to integrate a touch-sensitive interface into the therapeutic controller, enabling the user to select therapy modes, intensity, and duration while monitoring operational parameters and feedback.

It is also an object of the invention to provide a rechargeable, portable therapy system having internal power sources and charging ports, allowing convenient use and connection to external devices for recharging or coordinated operation.

Yet another object of the invention is to ensure neural pathway activation through coordinated stimulation of central and peripheral regions, wherein the timing, frequency, or phase of the first and second therapies are synchronized for enhanced bioelectrical or neuromuscular response.

A further object of the present invention is to provide a versatile therapeutic platform capable of functioning with different form factors such as handheld controllers, wearable masks, flexible pads, or caps, thereby accommodating various treatment needs, body regions, and user preferences.

### SUMMARY OF THE INVENTION

According to a first aspect of the present invention, a therapy system is provided. The therapy system comprises a first therapy device having one or more first stimulation elements configured to deliver a first therapy to a first treatment region of a user, and a therapeutic controller in communication with the first therapy device. The therapeutic controller comprises a user input interface configured to generate a control signal for operation of the first therapy device, and one or more second stimulation elements configured to deliver a second therapy to a second treatment region of the user. The second therapy may be delivered independently or concurrently with the first therapy, allowing for simultaneous or coordinated treatment across multiple body regions.

In one embodiment of the invention, the one or more first stimulation element and the one or more second stimulation element are selected from a group consisting of a phototherapy element, a microcurrent element, a pulsed electromagnetic field (PEMF) generator, an electromagnetic field (EMF) generator, an EMS element, a heating element, a cooling element, a Peltier element, a vibrator element, or a piezoelectric element.

In one embodiment of the invention, one or more first stimulation elements and the one or more second stimulation elements comprise a phototherapy unit including at least one light source having a plurality of lamp beads. The system may further include a lens assembly comprising a light-transmitting plate and a condenser lens aligned with the plurality of lamp beads for focused and uniform light emission.

In one embodiment of the invention, the housing of the therapeutic controller or therapy device further comprises a port configured to enable charging of an internal battery and connection to an external device. The port may also be configured to connect to an external phototherapy or stimulation device through a wired or wireless communication interface, thereby enabling cooperative operation or control.

In one embodiment of the invention, the therapeutic controller further comprises at least one physiological sensor selected from a temperature sensor, an impedance sensor, a motion sensor, a heart rate sensor, skin measuring sensor or a skin conductivity sensor. The controller may adaptively adjust delivery of the therapy based on feedback from the sensor, ensuring optimized stimulation intensity and safety.

In one embodiment of the invention, the user input interface comprises a touch-sensitive display configured to receive user input for selecting therapy mode, duration, or intensity. The interface may also display real-time physiological parameters and operational feedback.

According to a second aspect of the invention, a therapy system is provided comprising a first therapy device having one or more first stimulation elements configured to deliver a first therapy to a central nervous system (CNS) stimulation site of a subject, and a therapeutic controller in communication with the first therapy device. The therapeutic controller comprises a user input interface and at least one second stimulation element configured to deliver a second therapy to a peripheral stimulation site of the subject. The first therapy and the second therapy are delivered simultaneously or in a coordinated manner to activate a neural pathway extending between the CNS stimulation site and the peripheral stimulation site.

In one embodiment of the invention, the first and second therapies are synchronized in frequency, phase, or timing to enhance neural pathway activation and achieve synergistic therapeutic effects. The synchronization may be managed by the therapeutic controller using a timing algorithm or feedback loop.

In one embodiment of the invention, the therapeutic controller is further configured to operate the second stimulation element while transmitting a control signal to regulate delivery of the first therapy. In another embodiment, the therapeutic controller and the first therapy device are configured to alternate therapy delivery in a coordinated sequence to stimulate alternating neural or muscular responses.

In one embodiment of the invention, the therapeutic controller and the first therapy device may be connected wirelessly or via a wired interface, such that when connected, one device can act as a controller for the other. Both devices may provide therapy to different regions of the body simultaneously, enabling full-body or targeted multi-zone treatment.

According to a third aspect of the present invention, a method for providing one or more therapies to a user is provided. The method comprises delivering a first therapy to a treatment region using a first therapy device having one or more first stimulation elements, transmitting a control signal from a therapeutic controller to the first therapy device to regulate the first therapy, and delivering, concurrently or sequentially, a second therapy to the user using at least one second stimulation element integrated into the therapeutic controller.

In one embodiment of the invention, the method further comprises detecting a physiological parameter of the user using a sensor disposed in the therapeutic controller and adjusting at least one of the first or second therapies based on the detected physiological parameter.

In one embodiment of the invention, delivering the first therapy and the second therapy is performed simultaneously in a synchronized manner, allowing dual-region treatment and enhanced therapeutic efficiency through coordinated operation of the first and second stimulation elements.

The disclosed therapy system thus provides a multi-point stimulation architecture, where multiple therapeutic modalities such as light therapy, thermal therapy, or electrical stimulation can be applied independently or cooperatively to different body regions. This enables comprehensive treatment protocols for relaxation, rehabilitation, pain relief, or skin therapy, while maintaining compact form factors and intelligent control integration.

In the context of the specification, when an element is referred to as being “fixed to” or “disposed to” another element, it may either be directly on another element or indirectly on that other element. When a component is said to be “connected” or “connected to” another component, it may be directly connected to another component or indirectly connected to other components on the piece.

In the context of the specification, the terms “first”, “second,” and “third” are only used for descriptive purposes and do not imply the relative importance or implicitly indicate the quantity of technical features indicated.

In the context of the specification, the term “plurality” means two or more than two, unless otherwise indicated.

In the context of the specification, the term “several” means more than one, unless otherwise specified.

In the context of the specification, the term “LED module” refers to one or more light-emitting diode (LED) elements that are electrically connected and configured to emit light of specific wavelengths suitable for therapeutic purposes. The LED module may include drive circuitry, heat dissipation structures, and optical elements such as lenses or diffusers to control light distribution.

Unless otherwise stated, the term “light” as used in this specification encompasses electromagnetic radiation in the visible (380-780 nm) and infrared (780 nm-1000 nm) ranges, particularly red light (620-750 nm) and near-infrared (750-1400 nm) wavelengths commonly used in photobiomodulation therapy. Particular wavelengths which may be selected as the dominant emissive wavelength may include the follow, without any preference to be indicated by order: 400 nm, 405 nm, 420 nm, 430 nm, 450 nm, 465 nm, 515 nm, 530 nm, 532 nm, 590 nm, 630 nm, 633 nm, 640 nm, 650 nm, 655 nm, 660 nm, 670 nm, 680 nm, 780 nm, 785 nm, 810 nm, 830 nm, 840 nm, 850 nm, 860 nm, 870 nm, 904 nm, 915 nm, 980 nm, 1015 nm, 1060 nm, 1065 nm, 1070 nm, 1200, and 1400 nm. As used herein, the term “light therapy” refers to the use of one or more light sources of any type that emit light with a wavelength between about 400 and 1400 nm. The device may also emit blue or ultraviolet light for surface-level treatments such as acne reduction or microbial control.

The red light (approximately 630-660 nm) penetrates deeply into the scalp to stimulate blood circulation and enhance hair follicle activity, thus promoting hair growth and repair. Blue light (around 415-470 nm) exhibits antibacterial properties and is effective in treating scalp acne and reducing inflammation. Green light (approximately 520-540 nm) can help reduce pigmentation and soothe sensitive or irritated scalp tissue. Yellow light (around 580-600 nm) improves oxygen exchange in the cells and aids in detoxifying the scalp, while near-infrared light (800-850 nm) reaches deeper layers to accelerate healing and reduce pain.

In the context of this specification, terms like “light”, “radiation”, “irradiation”, “emission”, and “illumination”, etc., refer to electromagnetic radiation in frequency ranges varying from the visible frequencies to Infrared (IR) frequencies and wavelengths, wherein the range is inclusive of visible light, and IR frequencies and wavelengths. Preferably, it refers to low-level electromagnetic radiation of low-level red and near-infrared (NIR) light. It is to be noted here that IR radiation can be categorized into several categories according to respective wavelength ranges, which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm), and Far IR (15-1000 μm). In this regard, light application is at relatively low energy densities, typically below about 500 mW, as compared to other forms of laser therapy that are used for ablation, cutting, and thermally coagulating tissue. In some instances, electromagnetic radiation can also be in wavelengths in the blue or ultraviolet regions, especially for the treatment of conditions that occur at the skin surface, such as psoriasis or infection.

In the context of the specification, the term “light source” or “phototherapy source” etc. refers to a source emitting coherent laser light, or light-emitting diodes (“LEDs”). The term “light therapy” refers to light generated from any of the sources, such as lasers, LED sources, Super luminous diodes (“SLD”), or Organic light-emitting diodes (OLED).

In the context of the specification, “Light Emitting Diodes (LEDs)” refer to semiconductor diodes capable of emitting electromagnetic radiation when supplied with an electric current. The LEDs are characterized by superior power efficiencies, smaller sizes, rapid switching speeds, physical robustness, and longer lifespans compared to incandescent or fluorescent lamps. The one or more LEDs may include through-hole type LEDs (generally emitting electromagnetic radiation in red, green, yellow, blue, and white colors), Surface Mount Technology (SMT) LEDs, Bi-color LEDs, Pulse Width Modulated RGB (Red-Green-Blue) LEDs, and high-power LEDs, among others.

Materials used in one or more LEDs may vary from one embodiment to another, depending upon the frequency of radiation required. Different frequencies can be obtained from LEDs made from pure or doped semiconductor materials. Commonly used semiconductor materials include nitrides of Silicon, Gallium, Aluminum, Boron, Zinc Selenide, etc., in pure form or doped with elements such as Aluminum and Indium etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider color gamut. White and other colored lightings may also be produced using phosphor coatings such as Yttrium Aluminum Garnet (YAG) in combination with a blue LED to generate white light, and Magnesium-doped potassium fluorosilicate in combination with a blue LED to generate red light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety by reference.

In the context of the specification, the term “stimulation element” refers to may include, but is not limited to, phototherapy, micro-current, magneto therapy, cooling, heating, vibration, electrical pulses, or other forms of therapeutic output.

In the context of the specification, the term “electrotherapy,” “electrical stimulation,” or “microcurrent therapy” refers to the application of therapeutic current to the human body, regardless of the precise frequency, waveform, or current intensity.

In the context of the specification, the term “electrotherapy” may refer to different types of electrotherapy, including but not limited to galvanic, electrotherapy, iontophoresis, microcurrent, and EMS (Electrical Muscle Stimulation), TENS (Transcutaneous Electrical nerve stimulation).

In the case of a stimulation element being an electrode, the stimulation element may be embodied as an open-ended conductor. The electrode may then be able to provide Transcutaneous Electrical Nerve Stimulation (TENS), Electronic Muscle Stimulation (EMS), and Microcurrent Electrical Therapy (MET) to the target surfaces. TENS therapy uses low-voltage currents to provide pain relief. Electrical impulses are delivered through electrodes placed on the surface of the body of the user.

In the context of the present specification, the term “wearable housing” refers to any structure or assembly configured to support and position the phototherapy device on the body. The housing may be a facial mask, headband, wrap, patch, garment insert, or other ergonomically contoured body-conforming form factor. It may be rigid, semi-rigid, or flexible, and may be formed from materials such as silicone, polyurethane, polycarbonate, or textile composites.

In the context of the present specification, the term “micro-LED” refers to a semiconductor light-emitting device with a very small emission area, typically less than 200 μm per side, allowing for high pixel density, low power consumption, and precise control over wavelength and intensity. Micro-lasers in this context refer to miniature laser diodes or VCSELs (Vertical Cavity Surface Emitting Lasers) capable of emitting coherent light at therapeutic wavelengths.

In the context of the present specification, the term “connector” refers to any electrical interface, such as magnetic pogo pins, snap-fit contacts, or flexible flat cables, wires configured to couple the phototherapy device to an external controller, power source, or data interface. The connector may be integrated into the wearable housing and designed for quick attachment/detachment without tools.

In the context of the present specification, the term “heat dissipation” refers to design and material features that transfer or spread heat generated by the light sources to maintain skin-safe temperatures and prevent performance degradation. This may include high thermal conductivity substrates, thermally conductive encapsulants, or the metal-grid conductive structure acting as a heat-spreading layer.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings, wherein like reference letters and numerals indicate the corresponding parts in various figures in the accompanying drawings, and in which:

FIG. 1 illustrates a front perspective view of a controller, in accordance with an embodiment of the present invention.

FIG. 2 illustrates a back perspective view of the controller, in accordance with an embodiment of the present invention.

FIG. 3 illustrates an exploded view of the controller, in accordance with an embodiment of the present invention.

FIG. 4 illustrates a front perspective view of the lens component of the controller, in accordance with an embodiment of the present invention.

FIG. 5 illustrates a phototherapy mask in connection with the controller, in accordance with an embodiment of the present invention.

FIG. 6 illustrates an exploded view of the phototherapy mask, in accordance with an embodiment of the present invention.

FIG. 7 illustrates a perspective view of the phototherapy mask and the controller placed in a base device, in accordance with an embodiment of the present invention.

FIG. 8 illustrates a phototherapy cap in connection with the controller, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

The embodiment of the present invention provides a multifunctional controller device that itself constitutes a stimulation apparatus. The controller integrates within its housing a stimulation element which can provide microcurrent, EMS, phototherapy, ultrasonic wave therapy, etc. The housing accommodates a phototherapy light board and optical lens assembly configured to deliver focused therapeutic light directly to localized regions of the user's body. The controller further incorporates a circuit board, a rechargeable battery, and user interface elements such as keys and a display, thereby enabling independent operation as a standalone stimulation device. Through its compact structure and internal optical system, the controller is capable of providing portable, on-demand light therapy while simultaneously functioning as a hub for additional treatment modules.

In an embodiment, the invention provides a phototherapy mask ergonomically shaped to conform to the user's facial and neck regions. The mask comprises multiple phototherapy light boards arranged within inner and outer shells, with interlayers supporting arrays of light-emitting diodes (LEDs) or micro-LEDs for uniform irradiation of the skin. Protective eye masks and ventilation features improve comfort and safety, while snap-fit and positioning structures ensure stable assembly. The mask is electrically connectable to the controller, such that therapy modes and operating parameters can be adjusted through the controller's interface.

In an embodiment, the controller and mask are operable in conjunction, with the controller serving as a stimulation device, a power source, as well as a central control unit. The arrangement enables synchronized or independent activation of the controller's built-in stimulation device and the mask's facial/neck light panels, thereby providing comprehensive treatment options across multiple anatomical regions. The controller can be connected with a variety of stimulation devices/phototherapy devices, such as masks, pads, caps, etc. To further enhance convenience, a base unit is provided that accommodates both the controller and the mask when not in active use. The base incorporates spring-loaded electrical pins disposed within corresponding receiving slots, which engage the terminals of the controller and mask upon docking. Through these spring-pin connections, both the controller and mask batteries are automatically recharged while securely positioned in the base, ensuring readiness for subsequent therapeutic sessions.

The present invention relates to a multifunctional therapy system in which a first therapy device, such as a mask, pad, cap, or other wearable therapeutic unit, is configured to connect to a therapeutic controller capable of providing therapy independently or in coordination with the first device. The therapeutic controller not only regulates the operation of the first therapy device through wired or wireless communication but also incorporates its own stimulation elements, allowing it to deliver additional therapy to another region of the body. This integrated configuration enables simultaneous or alternating treatment across multiple areas, enhancing therapeutic efficiency and user convenience.

In an embodiment, the stimulation elements may be selected from a group comprising: a phototherapy element, a microcurrent element, a pulsed electromagnetic field (PEMF) generator, an electromagnetic field (EMF) generator, an electrical muscle stimulation (EMS) element, a heating element, a cooling element, a Peltier element, a vibrator element, or a piezoelectric element. Each of these elements may be used independently or in combination to achieve targeted therapeutic effects such as skin rejuvenation, muscle relaxation, neural stimulation, circulation enhancement, or pain relief.

In an embodiment of the present invention, the stimulation element in the controller and the first therapy device is a light therapy assembly. The light therapy assembly may include one or more light sources configured to emit light in therapeutic wavelength ranges. In some embodiments, the light sources are light-emitting diodes (LEDs) emitting red light, near-infrared light, or a combination thereof. Dual-wavelength or multi-wavelength arrays may be employed to provide enhanced treatment outcomes. The light output may be adjustable in terms of wavelength, intensity, and duration, with treatment modes tailored to specific therapeutic or cosmetic applications, such as skin rejuvenation, pain relief, muscle recovery, or circulation improvement.

In certain embodiments, the stimulation elements may include thermal stimulation, vibration stimulation, electrostimulation, or airflow-based stimulation, which can be selectively or jointly activated depending on the treatment mode.

The thermal stimulation may be provided by a heating element such as a resistive heater, a cooling element, or a thermoelectric Peltier module. The heating function can promote blood circulation, relax muscles, and improve absorption of cosmetic formulations, whereas the cooling function may reduce inflammation, soothe irritated skin, or provide localized analgesic relief. In some variations, the Peltier element may be reversible, thereby enabling both heating and cooling modes under electronic control.

The controller and the first therapy device may further include a vibration element, such as a miniature motor or actuator, configured to provide mechanical stimulation or massage during therapy. Vibration may aid in muscle relaxation, improve lymphatic drainage, and increase user comfort. In some cases, the vibration may be pulsed or modulated in intensity and may operate in synchronization with light or thermal therapy.

In another embodiment, the controller and the first therapy device may include an electrostimulation module designed to deliver controlled microcurrents to the skin surface. Such electrostimulation may activate muscles, improve skin tone, and complement the regenerative and circulatory benefits of light therapy. The electrostimulation circuit may be safeguarded with current regulators and safety cut-offs to ensure user safety.

In addition to phototherapy and thermal stimulation, the controller and the first therapy device may also provide electromagnetic stimulation. For example, the coil may be selectively driven to emit pulsed electromagnetic fields (PEMF) for therapeutic purposes. Such PEMF therapy may be offered independently or in combination with light therapy, providing synergistic wellness effects.

Airflow or pressure-based stimulation may also be incorporated, for example, through the use of a miniature fan or pneumatic element that directs cooling airflow or gentle air pulses onto the skin surface. This may enhance comfort during light therapy, reduce thermal buildup, or provide a refreshing treatment sensation.

These stimulation elements may be operated independently or in combination with the light therapy assembly to create synergistic effects. For instance, heating may be applied together with near-infrared light to increase tissue penetration, while cooling may be combined with blue light to improve acne treatment outcomes. Vibration may be synchronized with red or infrared light to encourage deeper relaxation of tissues. Treatment sequences may be preprogrammed within the control circuitry or may be customized by the user through an external mobile interface.

To ensure safe operation, each stimulation element may be paired with appropriate sensors, such as temperature sensors, contact sensors, or current sensors, along with automatic timers and safety cut-offs.

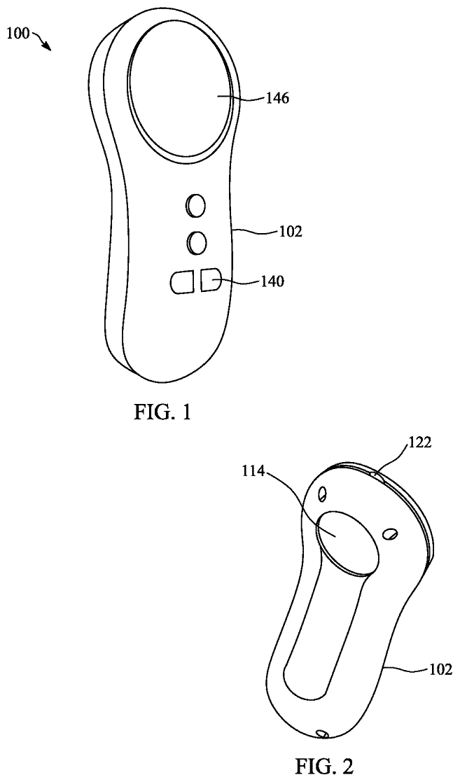

Referring to FIGS. 1 to 4, in an embodiment, a controller 100 is provided, comprising a housing 102 and one or more stimulation elements, such as a phototherapy assembly 104 disposed therein. The housing 102 defines, on its rear side, a first positioning hole 106 configured to accommodate optical components.

In an embodiment, the one or more stimulation elements may be selected from a group comprising: a phototherapy element, a microcurrent element, a pulsed electromagnetic field (PEMF) generator, an electromagnetic field (EMF) generator, an electrical muscle stimulation (EMS) element, a heating element, a cooling element, a Peltier element, a vibrator element, or a piezoelectric element. Each of these elements may be used independently or in combination to achieve targeted therapeutic effects such as skin rejuvenation, muscle relaxation, neural stimulation, circulation enhancement, or pain relief.

In an embodiment, the one or more stimulation elements comprises a phototherapy unit including one or more light sources with a plurality of LED lamp beads capable of emitting light at multiple wavelengths, such as red, blue, green, violet, yellow, near-infrared, or white light. These light sources are configured to perform wavelength-specific phototherapy tailored to different skin or tissue needs. A lens assembly may be provided in alignment with the plurality of lamp beads, the lens assembly including a light-transmitting plate and a condenser lens for focusing and evenly distributing the emitted light toward the treatment region. This structure ensures uniform irradiation intensity and improves the therapeutic efficiency of the phototherapy process.

The stimulation element shown is a phototherapy assembly 104 that comprises a circuit board 108, a phototherapy light board 110, and a lens assembly 112, all of which are arranged within the housing 102. The circuit board 108 is electrically connected to the phototherapy light board 110, which is adapted to control the operating state of the lamp beads mounted on the phototherapy light board 110, including their activation, deactivation, and light intensity adjustment. The phototherapy light board 110 is positioned between the circuit board 108 and the lens assembly 112, such that the surface of the light board facing the lens assembly 112 is electrically connected to a plurality of lamp beads.

The lens assembly 112 includes a light-transmitting plate 114 and a lens body 116. The light-transmitting plate 114 is fixed within the first positioning hole 106 of the housing 102 and serves as the final exit plane for light emitted from the device. The lens body 116 is disposed on the side of the light-transmitting plate 114 facing the phototherapy light board 110 and is positioned in correspondence with each lamp bead. This arrangement ensures that the emitted light from each lamp bead is effectively focused and directed through the light-transmitting plate 114 toward the treatment area.

In operation, when the device is activated by a user, the circuit board 108 delivers a control signal to the phototherapy light board 110, thereby driving the lamp beads to emit light of predetermined wavelengths suitable for beauty and skin-care applications. Because each lamp bead is optically aligned with a corresponding lens body 116, the emitted light is concentrated and collimated. The focused light then passes through the light-transmitting plate 114 and illuminates the target treatment region, such as the facial skin or another body part. This optical arrangement significantly enhances the density of light energy delivered to the skin surface, thereby improving light utilization efficiency, enhancing therapeutic effect, and overcoming the problems of uneven illumination and poor efficacy associated with traditional phototherapy devices.

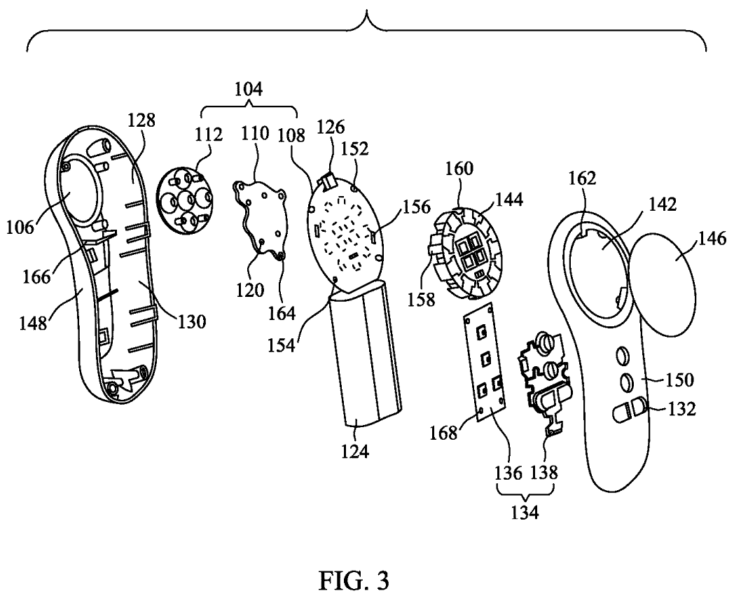

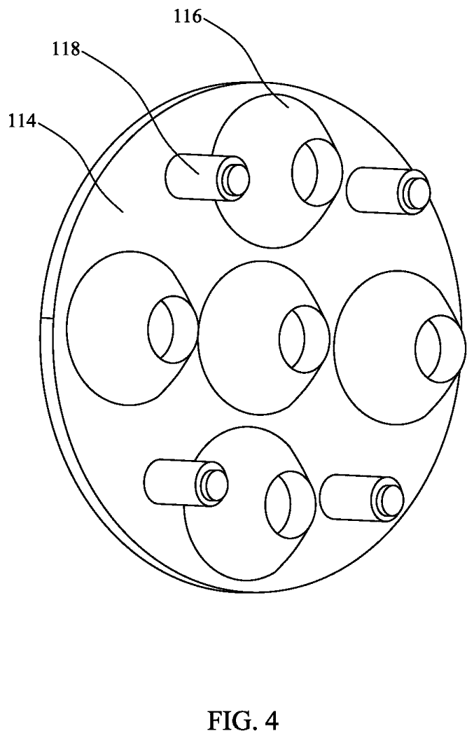

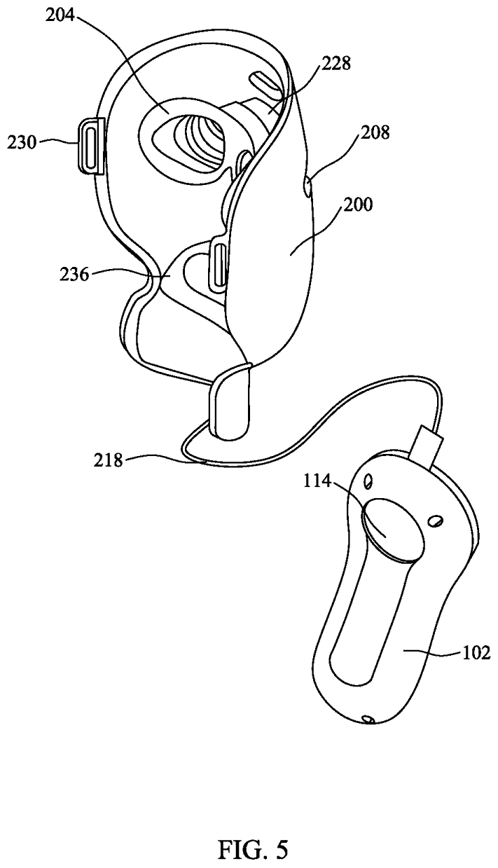

Referring to FIGS. 3 and 4, the lens assembly 112 additionally comprises at least one limiting post 118. The limiting post 118 is disposed on the side of the light-transmitting plate 114 facing the phototherapy light board 110. The phototherapy light board 110 is correspondingly formed with at least one limiting hole 120 for receiving the limiting post 118. In one exemplary configuration, four limiting posts 118 may be provided, one at each corner of the light-transmitting plate 114, and the phototherapy light board 110 is provided with four corresponding limiting holes 120.

The engagement between the limiting posts 118 and limiting holes 120 provides a reliable reference and positioning structure, ensuring accurate alignment between the light-transmitting plate 114 and the phototherapy light board 110. This arrangement facilitates quick assembly and secure fixation of the optical components, thereby maintaining stable relative positioning of the light-transmitting plate 114 and the phototherapy light board 110 during operation. Precise alignment of the lens body 116 with the lamp beads on the phototherapy light board 110 is thus achieved, ensuring consistent and effective focusing of light and thereby enhancing the overall phototherapy performance of the device.

In an embodiment, the lens assembly 112 is configured as an integrally molded structure. In particular, the lens assembly 112 may be manufactured by injection molding, die-casting, or other integral molding processes. The use of integral molding eliminates the need for separate fabrication and assembly of multiple subcomponents, thereby reducing manufacturing complexity and simplifying the overall assembly process. The integrated design further enhances the structural strength and stability of the lens assembly 112, ensuring durability and reliability during repeated use.

In an embodiment, and with reference to FIGS. 3 and 4, the lens body 116 of the lens assembly 112 is formed such that its cross-sectional area gradually increases in the direction extending from the phototherapy light board 110 toward the light-transmitting plate 114. In this arrangement, the lens body 116 narrows at the end proximate to the lamp beads and widens toward the end adjacent to the light-transmitting plate 114. The shape of the lens body 116 may, for example, approximate a truncated cone or trumpet form. This gradual expansion of the cross-sectional area functions to both concentrate light emitted from the lamp beads and, at the same time, progressively broaden the illumination range. As a result, light energy is evenly distributed over a wider treatment area, thereby improving light utilization efficiency and avoiding localized overheating or non-uniform illumination. This ensures efficient light transmission and promotes a more uniform distribution of therapeutic light across the target surface.

In an embodiment, the therapeutic controller further comprises one or more physiological sensors integrated into the housing. These sensors may include, but are not limited to, a temperature sensor, impedance sensor, motion sensor, heart rate sensor, skin measuring sensor, or skin conductivity sensor. These sensors are configured to monitor physiological parameters of the user during therapy sessions, allowing the system to dynamically adjust therapy delivery based on real-time feedback. For example, when the detected skin temperature exceeds a predetermined threshold, the system may reduce the output intensity of the heating or light-emitting elements to maintain user safety and comfort.

Referring to FIGS. 2 and 3, in an embodiment, the housing 102 of the phototherapy device is provided with a connector 122. A rechargeable battery 124 is disposed within the housing 102 and is electrically connected to the circuit board 108. The circuit board 108 is further provided with a port 126 corresponding to the connector 122. The port 126 is configured both to enable charging of the rechargeable battery 124 and to provide an electrical connection to an external phototherapy mask.

In an embodiment, the port 126 of the circuit board 108 may be implemented as a Type-C port, thereby allowing a single interface to support both charging functions and external device connections. This dual-purpose configuration simplifies the structural design of the device, reduces the number of required interfaces, and enhances user convenience. The circuit board 108 manages charging of the rechargeable battery 124 and is further adapted to selectively control the operation of the internal phototherapy light board 110 and an externally connected phototherapy mask. For example, when charging is required, a Type-C charging cable may be inserted into the port 126 through the connector 122 of the housing 102, enabling recharging of the rechargeable battery 124. When facial phototherapy is desired, the user may connect the phototherapy mask to the port 126, whereupon the circuit board 108 activates the external mask while deactivating the internal phototherapy light board 110. Furthermore, when simultaneous phototherapy of both the face and another body part is required, the circuit board 108 may be configured to operate both the external mask and the internal phototherapy light board 110 concurrently, with the user selecting the desired treatment modes through the control panel. Through these structural and functional features, the present invention provides a compact, multifunctional device capable of delivering phototherapy to multiple treatment areas with improved efficiency and convenience.

According to an embodiment of the present invention, when the user requires phototherapy for a localized body region, the phototherapy mask may be disconnected from the device, whereupon the phototherapy light board 110 is activated and an appropriate phototherapy mode is selected via the circuit board 108. In this configuration, the device operates independently to deliver body phototherapy.

In a representative implementation, the phototherapy mask may be configured with nine distinct phototherapy modes, including: red light combined with near-infrared light, red light alone, blue light, green light, near-infrared light, violet light, yellow light, deep cyan light, and white light. The phototherapy light board 110, in comparison, may be configured with five distinct phototherapy modes, such as red light, blue light, green light, near-infrared light, and violet light. It should be understood that the specific number and type of phototherapy modes available to the mask and the light board are not limited to the enumerated examples, and in alternative embodiments, the phototherapy mask and the phototherapy light board 110 may be provided with identical sets of phototherapy modes. The exact configuration may be determined based on design considerations and user requirements.

By providing both an internal phototherapy light board 110 and an external phototherapy mask, the device of the present invention is capable of offering localized body treatment via the phototherapy light board 110 and facial treatment via the mask. This dual arrangement expands the application range of the device and significantly enhances its versatility, enabling it to meet diverse phototherapy needs across different body regions.

Referring to FIGS. 2 and 3, in an embodiment, the housing 102 of the controller device is internally divided into a first chamber 128 and a second chamber 130, which are arranged vertically and interconnected. The first positioning hole 106 and the connector 122 are both in communication with the first chamber 128. The phototherapy assembly 104 is disposed within the first chamber 128, while the rechargeable battery 124 is accommodated within the second chamber 130.

In an embodiment, the device adopts a layered structural design wherein functional components are segregated into different chambers. The upper first chamber 128 is primarily responsible for performing phototherapy functions, whereas the lower second chamber 130 houses the power supply components. This separation provides electrical isolation between the power supply and control circuitry, thereby improving operational safety. Moreover, the compartmentalized layout allows for efficient utilization of space, enhances structural compactness, and supports overall device miniaturization.

Referring to FIG. 1, and 3, a second positioning hole 132 is defined on the front side of the housing 102, in communication with the second chamber 130. A key module 134 is disposed within the second chamber 130 and comprises a driver board 136 and a key structure 138. The driver board 136 is located between the rechargeable battery 124 and the key structure 138 and is electrically connected to the circuit board 108. The key structure 138 is electrically coupled to the side of the driver board 136 opposite the rechargeable battery 124 and is mounted within the second positioning hole 132.

In this configuration, the key structure 138 includes a plurality of keys 140, which are exposed on the front surface of the housing 102 through the second positioning hole 132, thereby allowing direct user operation. The second positioning hole 132 facilitates precise alignment and quick installation of the key structure 138 during assembly, improving manufacturing efficiency and reliability. During use, actuation of a key 140 generates a mechanical action signal that is converted into a digital control signal by the driver board 136 and transmitted to the circuit board 108. Upon receipt of the signal, the circuit board 108 adjusts the operational parameters of the phototherapy light board 110, including power state, light intensity, wavelength mode, and treatment duration. When an external phototherapy mask is connected, the circuit board 108 may additionally regulate its operating parameters.

This design of the key module 134 provides an intuitive, user-friendly control interface while maintaining compactness and simplicity in the overall structure. The arrangement ensures that users can conveniently operate and adjust the phototherapy device with precision, while the internal layered layout preserves functional integration and miniaturization.

Referring to FIGS. 1 to 3, in an embodiment, a through-hole 142 is formed on the front side of the housing 102 and is in communication with the first chamber 128. A display screen 144 is disposed within the first chamber 128 and electrically connected to the circuit board 108 on the side opposite the phototherapy light board 110. The position of the display screen 144 corresponds to the through-hole 142, such that the displayed content is clearly visible therethrough. The display screen 144 provides the user with real-time operational information of the light therapy device, including the selected light therapy mode, remaining treatment time, and battery status. This arrangement facilitates intuitive operation and improves overall user experience.

In an embodiment, a protective film 146 is provided within the through-hole 142 to cover the display screen 144. The protective film 146 may be formed of a transparent, scratch-resistant, and anti-reflective material, such as a polyester film. The protective film 146 effectively isolates the display screen 144 from the external environment, thereby reducing the likelihood of physical damage or contamination and extending the service life of the display screen 144.

The housing 102 comprises a bottom housing 148 and a cover shell 150. The bottom housing 148 is provided with a first positioning hole 106, while the cover shell 150 is provided with a second positioning hole 132 and the through-hole 142. A first notch is formed at the top of the bottom housing 148, and a second notch is formed at the top of the cover shell 150. When assembled, the first notch and second notch together define a connector 122. This structural configuration facilitates the installation of internal components, thereby improving assembly efficiency and maintenance convenience of the light therapy device.

In one embodiment, snap rings are provided on both sides of the bottom housing 148, while corresponding first snap rings are provided on both sides of the cover shell 150. The first snap rings engage with the snap rings, allowing the bottom housing 148 and cover shell 150 to be removably assembled.

In an embodiment, first connecting posts are arranged at the four corners of the bottom housing 148, and second connecting posts are correspondingly arranged at the four corners of the cover shell 150. The circuit board 108 is provided with two first connecting holes 152, which align with the two first connecting posts located at the upper portion of the bottom housing 148. Additionally, two-thirds of the connecting posts are disposed in the cover shell 150 near the through-hole 142, and the circuit board 108 is provided with two second connecting holes 154 corresponding to the third connecting posts.

During assembly, screws are inserted through the second connecting holes 154 of the circuit board 108 and fastened to the third connecting posts of the cover shell 150. Further, screws are inserted through the first connecting posts of the bottom housing 148, the two first connecting holes 152 of the circuit board 108, and into the second connecting posts of the cover shell 150, thereby securely fixing the circuit board 108 within the housing 102.

According to an embodiment of the present invention, as illustrated in FIG. 3, the circuit board 108 is provided with one or more snap-fit holes 156. A second snap 158 is formed on the edge of a display screen 144. The second snap 158 engages with the snap-fit hole 156, thereby securely attaching the display screen 144 to the circuit board 108. In certain embodiments, two snap-fit holes 156 may be provided to enhance stability.

Further, a retaining groove 160 is formed along the top edge of the display screen 144, and a corresponding retaining protrusion 162 is provided on the through-hole 142 of the cover shell 150. During assembly, when the display screen 144 is mounted within the through-hole 142, the retaining protrusion 162 engages with the retaining groove 160, thereby securing the display screen 144 in its intended position within the housing.

In an embodiment, a plurality of fourth connecting posts are provided within the first chamber 128 of the bottom housing 148. These fourth connecting posts are spaced circumferentially around the first positioning hole 106. The phototherapy light board 110 includes corresponding third connecting holes 164 along its edge, which align with the fourth connecting posts. For example, three-fourths of connecting posts may be provided. During assembly, the light-transmitting plate 114 is first installed within the first positioning hole 106. The phototherapy light board 110 is then positioned over the light-transmitting plate 114 using the limiting posts 118. Screws are subsequently inserted through the third connecting holes 164 of the phototherapy light board 110 and engaged with the fourth connecting posts of the bottom housing 148, thereby securing both the light-transmitting plate 114 and the phototherapy light board 110 in place.

Referring to FIG. 3, additionally, a partition 166 is provided within the bottom housing 148, effectively dividing the interior of the housing 102 into a first chamber 128 and a second chamber 130.

Moreover, four-fifths of the connecting posts and two stopper plates are provided within the cover shell 150. The four-fifths of the connecting posts are positioned at the four corners of the key structure 138, while the two stopper plates are located on opposite sides of the key structure 138. The driver board 136 is provided with fourth connection holes 168 corresponding to the fifth connecting posts. During assembly, the driver board 136 is placed over the key structure 138 and positioned using the stopper plates such that the fourth connection holes 168 align with the corresponding fifth connecting posts. The assembly is then secured with screws, thereby fixing the key module 134 within the cover shell 150.

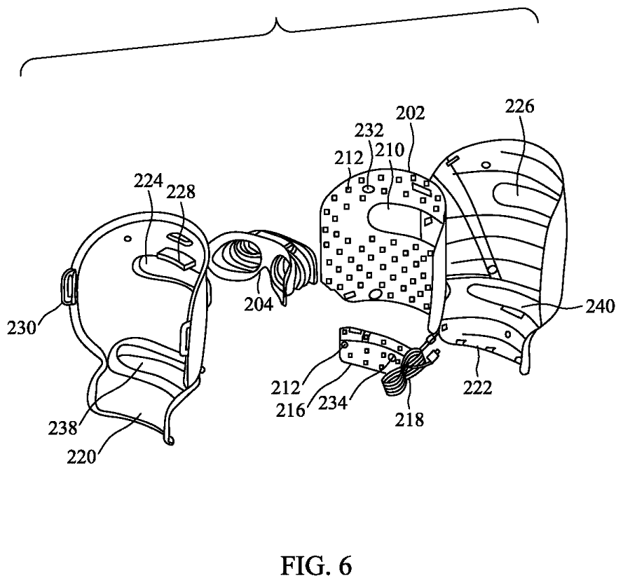

According to another embodiment of the present invention, referring to FIGS. 5 and 6, a phototherapy mask 200 is provided, comprising a first phototherapy light board 202 and an eye mask 204. The phototherapy mask 200 is configured with a first interlayer corresponding to the contour of the face and a first through-hole 208 positioned at a location corresponding to the eye. The first phototherapy light board 202 is accommodated within the first interlayer and is provided with a second through-hole 210, which is aligned with and communicates with the first through-hole 208. The light-emitting surface of the first phototherapy light board 202 is oriented toward the inner side of the phototherapy mask 200, adjacent to the wearer's face. The eye mask 204 is inserted through the first through-hole 208 and the second through-hole 210, extending toward the inner side of the phototherapy mask 200 in proximity to the face. The eye mask 204 serves to create a gap between the phototherapy mask 200 and the wearer's face.

In this embodiment, the phototherapy mask 200 functions as the primary structural base of the device and is designed to cover the user's face during operation. The phototherapy mask 200 incorporates the first interlayer for securely mounting the first phototherapy light board 202. The first through-hole 208 is positioned at the area corresponding to the user's eye, thereby allowing the wearer to retain a clear field of vision during use and facilitating the placement of the eye mask 204.

The first phototherapy light board 202, disposed within the first interlayer of the phototherapy mask 200, is provided with the second through-hole 210. The second through-hole 210 is precisely aligned with the first through-hole 208 of the phototherapy mask 200 to prevent direct light emission toward the eyes. The light-emitting side of the first phototherapy light board 202 is equipped with LED beads 212, or other suitable light sources, capable of emitting light at predetermined wavelengths for phototherapy and skincare applications. When the device is activated, the first phototherapy light board 202 emits therapeutic light that penetrates the phototherapy mask 200 and irradiates the user's facial skin, thereby promoting blood circulation and enhancing metabolic activity.

The eye mask 204 is positioned on the inner side of the phototherapy mask 200 and extends through the first through-hole 208 and the second through-hole 210. The eye mask 204 performs dual functions: it shields the user's eyes from direct light exposure and provides spacing between the phototherapy mask 200 and the user's face. This spacing facilitates heat dissipation and ventilation, thereby improving air circulation within the mask and reducing thermal buildup. As a result, overall user comfort is significantly enhanced during prolonged use of the phototherapy mask. This also increases the distance between the first phototherapy light board 202 and the skin, thereby ensuring a more uniform distribution of light. As can be observed, the provision of the eye mask 204 not only shields the eyes from strong light during phototherapy but also maintains a clear field of vision for the wearer. Moreover, the gap formed between the phototherapy mask 200 and the face enhances ventilation and air permeability in the local region, reducing the risk of temperature rise caused by prolonged contact and thereby minimizing the potential for heat-related damage. Consequently, the phototherapy mask 200 according to this embodiment of the present invention effectively enhances user comfort and overall experience.

In an embodiment, the phototherapy mask 200 further comprises a second interlayer positioned to correspond to the neck, which is integrally connected to the first interlayer. The phototherapy mask 200 further includes a second phototherapy light board 216, disposed within the second interlayer, with its light-emitting surface facing the inner side of the phototherapy mask 200 adjacent to the neck region. The second phototherapy light board 216 is electrically connected to the first phototherapy light board 202.

In an embodiment, the second interlayer is connected to the first interlayer to form a unified structure, ensuring an integrated design of the device while enabling simultaneous facial and neck treatment. The second phototherapy light board 216, mounted within the second interlayer, corresponds to the user's neck area and includes LED beads 212 or other suitable light sources configured to emit light at a specific therapeutic wavelength. The second phototherapy light board 216, is specifically designed to provide phototherapy for neck-related skin concerns, such as wrinkles. The first phototherapy light board 202 and the second phototherapy light board 216 are electrically interconnected and may be controlled either simultaneously or independently, allowing adjustment of therapy modes as required to meet different skincare needs. Thus, by incorporating a neck care function, this embodiment of the present invention not only improves the therapeutic effect but also broadens its application scenarios, thereby further enhancing the user experience.

According to an embodiment of the present invention, the phototherapy mask 200 additionally comprises a wire 218. One end of the wire 218 is electrically connected to either the first phototherapy light board 202 or the second phototherapy light board 216, while the other end of the wire 218 extends through the phototherapy mask 200 and is adapted to be electrically connected to a controller.

In an embodiment, the wire 218 functions as a critical component for electrically coupling the light panels to the external controller. One end of the wire 218 is directly connected to the light panels disposed within the phototherapy mask 200, while the other end passes through the phototherapy mask 200 to establish an external connection with the controller 100. This configuration enables the user to remotely operate the device via the controller 100. The controller 100 is equipped with a display screen, control buttons, stimulation element, and other components, thereby allowing users to conveniently select light therapy modes, adjust treatment duration, and configure additional operational parameters.

The user input interface of the controller 100 may be implemented as a touch-sensitive display panel, enabling the user to select therapy parameters such as mode, duration, wavelength, or intensity. The display may also present real-time data regarding system operation, battery level, or sensor feedback.

For example, the light therapy modes include nine selectable options: red light plus near-infrared light, red light, blue light, green light, near-infrared light, violet light, yellow light, deep cyan light, and white light. By default, the duration for each mode is 10 minutes, though users may adjust this duration according to individual requirements. It is understood that both the first phototherapy light board 202 and the second phototherapy light board 216 may operate under any of the above modes. Through the controller, users may choose to activate the first phototherapy light board 202 and the second phototherapy light board 216 either independently or simultaneously, thereby enabling customized and comprehensive phototherapy plans tailored to personal needs.

In an embodiment, the phototherapy mask 200 comprises a connected inner shell 220 and outer shell 222. The first interlayer and the second interlayer are located between the inner shell 220 and the outer shell 222. The inner shell 220 defines a first cut-out 224 corresponding to the eye area, and the outer shell 222 defines a second cut-out 226 corresponding to and communicating with the first cut-out 224. The first through-hole 208 is thus composed of the first cut-out 224 and the second cut-out 226. The light-emitting surfaces of both the first phototherapy light board 202 and the second phototherapy light board 216 face the inner shell 220, while the backlight sides of the first phototherapy light board 202 and the second phototherapy light board 216 (i.e., the surfaces facing away from the LED beads 212) are directed toward the outer shell 222. The eye mask 204 is positioned on the inner side of the inner shell 220 adjacent to the face and extends through the first cut-out 224, the second through-hole 210, and the second cut-out 226, thereby creating a gap between the inner shell 220 and the user's face.

In an embodiment, the eye mask 204 is mounted on the inner side of the inner shell 220 and extends sequentially through the first cut-out 224 of the inner shell 220, the second through-hole 210 of the first phototherapy light board 202, and the second cut-out 226 of the outer shell 222. This arrangement not only prevents direct light exposure to the user's eyes but also ensures a consistent spacing between the inner shell 220 and the face via the eye mask 204, thereby improving user comfort and facilitating air circulation within the mask.

In an embodiment of the present invention, a first positioning structure is provided on the inner side of the inner shell 220 adjacent to the face, while a corresponding second positioning structure is provided on the eye mask 204. One of the first and second positioning structures comprises a positioning protrusion 228, and the other comprises a corresponding positioning groove. The positioning protrusion 228 aligns with the positioning groove.

In an embodiment, the first positioning structure is located on the inner side of the inner shell 220 adjacent to the face and may be formed as either the positioning protrusion 228 or the positioning groove. The second positioning structure, located on the eye mask 204, is configured to correspond to the first positioning structure. For example, in one implementation, the inner shell 220 is provided with the positioning protrusion 228, while the eye mask 204 is provided with the positioning groove. When the eye mask 204 is placed inside the inner shell 220, the positioning protrusion 228 engages with the positioning groove, thereby ensuring accurate positioning and stable fixation of the eye mask 204. This arrangement prevents displacement of the eye mask 204 during use, thereby avoiding the risk of direct light exposure to the eyes and enhancing safety during operation.

By ensuring that the eye mask 204 remains fixed in position, discomfort caused by shifting or misalignment can be minimized. In addition, accurate positioning of the eye mask 204 provides an improved field of vision, allowing the user to open their eyes without obstruction while receiving phototherapy. The positioning structure also prevents deformation of the eye mask 204 under stress and maintains an appropriate distance between the inner shell 220 and the face. This ensures uniform illumination, effective ventilation, and proper heat dissipation, thereby improving stability and enhancing the comfort of the wearer.

In an embodiment, the edge of the inner shell 220 is provided with more than twenty strap buckles 230. In this embodiment, the strap buckles 230 may be distributed at different positions along the edge of the inner shell 220, such as on the sides, top, or bottom, thereby ensuring an even distribution of tension. This arrangement enables the phototherapy mask 200 to be securely and comfortably worn on the user's face with the aid of straps (not shown). The strap may be fabricated from either elastic or non-elastic material, with one end fixed to the strap buckle 230. The other end of the strap may be adjustable in length to accommodate different head sizes.

In an embodiment, the side of the inner shell 220 adjacent to the first phototherapy light board 202 and the second phototherapy light board 216 is provided with multiple first snap-fit structures, while the side of the outer shell 222 adjacent to the first phototherapy light board 202 and the second phototherapy light board 216 is provided with multiple second snap-fit structures. The first snap-fit structures and the second snap-fit structures correspond in a one-to-one manner. Each pair of first snap-fit structure and second snap-fit structure consists of one snap-fit and one slot, with the snap-fit engaging the slot. Specifically, the plurality of first snap-fit structures of the inner shell 220 and the plurality of second snap-fit structures of the outer shell 222 may be flexibly configured as all snap-fits, all slots, or a combination of snap-fits and slots, depending on assembly requirements. Each first snap-fit structure securely engages with the corresponding second snap-fit structure to form a stable connection. This snap-fit mechanism enables convenient assembly of the inner shell 220 and outer shell 222, stabilizes the first phototherapy light board 202, ensures uniform light distribution, and facilitates disassembly for maintenance or replacement of the first phototherapy light board 202.

In an embodiment, a plurality of third snap-fit structures are provided on the side of the inner shell 220 adjacent to the second phototherapy light board 216. Correspondingly, a plurality of fourth snap-fit structures are arranged on the side of the outer shell 222 adjacent to the second phototherapy light board 216. The third snap-fit structures and the fourth snap-fit structures correspond in a one-to-one manner and are disposed around the second phototherapy light board 216. Each pair of third snap-fit structures and fourth snap-fit structures consists of one latch and one slot, with the latch engaging the slot. Similar to the first snap-fit configuration, the plurality of third snap-fit structures and fourth snap-fit structures may be arranged as all latches, all slots, or a combination thereof, depending on specific assembly needs. Each third snap-fit structure engages with its corresponding fourth snap-fit structure to establish a secure connection. This snap-fit method allows quick and convenient assembly of the inner shell 220 and outer shell 222, ensuring stability of the second phototherapy light board 216, uniform illumination, and easy disassembly for maintenance or replacement.

In an embodiment, the inner shell 220 defines a first connection hole and a second connection hole. The first phototherapy light board 202 defines a third connection hole corresponding to the first connection hole. The second phototherapy light board 216 defines a fourth connection hole 234 corresponding to the second connection hole. A first connection post is disposed on the side of the outer shell 222 adjacent to the first phototherapy light board 202. The first connection post is connected to the first connection hole and the third connection hole 232 via a first fastener (not shown). Similarly, a second connection post is disposed on the side of the outer shell 222 adjacent to the second phototherapy light board 216. The second connection post is connected to the second connection hole and the fourth connection hole 234 via a second fastener.

For example, the inner shell 220 may be provided with four first connection holes, positioned at the four corners of the first interlayer. The second connection hole may be a single hole located at the center of the second interlayer. Accordingly, the first phototherapy light board 202 may include four third connection holes 232, the second phototherapy light board 216 may include one fourth connection hole 234, and the outer shell 222 may include four first connection posts and one second connection post. During assembly of the phototherapy mask, a first fastener (e.g., a screw) penetrates the first connection holes of the inner shell 220 and the third connection holes 232 of the first phototherapy light board 202, and is secured to the first connection posts of the outer shell 222, thereby fixing the first phototherapy light board 202. Likewise, a second fastener (e.g., a screw) penetrates the second connection hole of the inner shell 220 and the fourth connection hole 234 of the second phototherapy light board 216, and is secured to the second connection post of the outer shell 222, thereby fixing the second phototherapy light board 216. This assembly forms a robust and integrated structure, ensuring that the inner shell 220, outer shell 222, and light panels remain stable and reliable during use.

In an embodiment, a wire hole is provided on the edge of the inner shell 220. This hole allows the wire 218 to pass through the phototherapy mask 200 and connect with an external controller 100.

In an embodiment, the phototherapy mask 200 further includes a positioning hole 236 corresponding to the user's chin. The positioning hole 236 is disposed between the first phototherapy light board 202 and the second phototherapy light board 216, corresponding to the chin area. The positioning hole 236 may be formed in a strip shape to ensure a comfortable and secure fit. The inner shell 220 of the phototherapy mask 200 is provided with a first hole 238, while the outer shell 222 of the phototherapy mask 200 is provided with a second hole 240 corresponding to and aligned with the first hole 238. Together, the first hole 238 and the second hole 240 constitute the positioning hole 236 of the phototherapy mask 200.

The positioning hole provides an additional ventilation opening for the user's face, thereby reducing stuffiness and improving comfort during extended use of the phototherapy mask 200. By placing the chin into the positioning hole, the user can quickly and accurately position the phototherapy mask 200. This intuitive design reduces adjustment time, simplifies the wearing process, and enhances user convenience. Thus, the provision of the chin positioning hole not only improves breathability and comfort but also optimizes overall user experience.

The positioning hole 236 serves a dual function: it provides an additional ventilation opening for the face, thereby reducing stuffiness and improving comfort during extended use, and it enables the user to quickly align the chin with the mask for proper wearing. This intuitive design reduces adjustment time and simplifies the wearing process. Thus, the inclusion of the positioning hole 236 not only enhances breathability and comfort but also improves usability and overall user experience.

In an embodiment, the phototherapy mask 200 comprises an integrally formed and connected first phototherapy portion, a transition portion, and a second phototherapy portion. The first phototherapy portion includes the first interlayer for mounting the first phototherapy light board 202 to provide facial phototherapy. The transition portion connects the first phototherapy portion and the second phototherapy portion and is provided with the positioning hole 236. The second phototherapy portion includes the second interlayer for mounting the second phototherapy light board 216 to provide neck phototherapy. This arrangement enhances the compactness and integrity of the mask structure and optimizes internal space utilization. The phototherapy mask 200 is formed as a hard component. For example, the phototherapy mask 200 may be manufactured from hard silicone, plastic, or other rigid materials to improve structural stability, durability, and portability. The eye mask 204 is formed as a flexible component. For example, the eye mask 204 may be fabricated from flexible silicone or rubber material to enhance wearing comfort and adaptability.

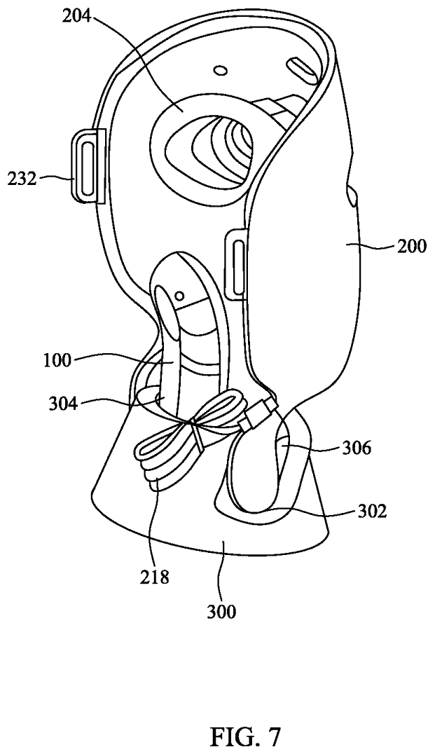

According to one embodiment of the present invention, FIG. 7 illustrates a phototherapy device comprising a base 300, a phototherapy mask 200, and a controller 100.

The base 300 serves as a supporting portion for the entire device and is provided with a slot 302 and a socket 304. The slot 302 is configured for the installation of the phototherapy mask 200, while the socket 304 is configured for the installation of the controller 100.

The controller 100 comprises a housing, a control board, and a phototherapy component, which are arranged within the housing. The housing is configured to be detachably inserted into the socket 304 of the base 300. The control board is electrically connected to the phototherapy mask 200, and the phototherapy component is electrically connected to the control board.

The phototherapy mask 200 is designed to be detachably mounted in the slot 302 of the base 300. The phototherapy mask 200 comprises a plurality of light-emitting diodes (LEDs) capable of emitting light of specific wavelengths to perform phototherapy on the facial skin. The phototherapy mask 200 can be easily removed from the slot 302 and electrically connected to the controller 100.

The housing of the controller 100 accommodates the control board and the phototherapy component, which can be used to irradiate a local area of the body, such as the hands or legs. The controller 100 is detachably secured to the base 300 via the socket 304 and can function either independently or in conjunction with the phototherapy mask 200 through electrical connection.

When a user requires phototherapy for the face, the phototherapy mask 200 is removed from the base 300, electrically connected to the controller 100, and a desired phototherapy mode for the phototherapy mask 200 is selected through the control board. In this state, the phototherapy component of the controller 100 remains inactive. When phototherapy is required for a localized area of the body, the controller 100 can be removed from the base 300, and a desired phototherapy mode for the phototherapy component can be selected via the control board, while the phototherapy mask 200 remains inactive.

Further, when the user desires simultaneous phototherapy for both the face and another body area, the phototherapy mask 200 and the controller 100 may both be removed from the base 300 and electrically connected. The user can then select respective phototherapy modes for the phototherapy mask 200 and the phototherapy component via the control board.

In an embodiment, the phototherapy mask 200 supports nine phototherapy modes, including: red light combined with near-infrared light, red light, blue light, green light, near-infrared light, violet light, yellow light, dark cyan light, and white light. The phototherapy component of the controller 100 supports five phototherapy modes, including red light, blue light, green light, near-infrared light, and violet light. It is also possible for both components to operate under the same phototherapy mode, depending on the user's actual requirements.

Accordingly, the phototherapy device provided by this embodiment of the present invention enables phototherapy for both the face and specific parts of the body, providing enhanced flexibility and allowing users to select suitable phototherapy methods based on personal skincare needs. The device can thus accommodate both localized treatment and comprehensive multi-area therapy.

In an embodiment, the phototherapy mask 200 comprises a mask body electrically connected to the control board. The mask body includes a first phototherapy section and a second phototherapy section integrally connected thereto. The first phototherapy section is configured for phototherapy of the face, while the second phototherapy section is configured for phototherapy of the neck. The second phototherapy section is inserted into the slot 302 of the base 300, thereby ensuring stable mounting of the mask body on the base 300.

In another embodiment of the present invention, referring to FIG. 7, a base 300 is provided with a slot 302 and a socket 304, holding a phototherapy mask 200 and a controller 100. The outer wall of the slot 302 of the base 300 defines a socket. A receiving cavity is formed within the base 300 and communicates with the socket. A retaining plate is provided on the side of the cavity facing away from the socket. The elastic pad 306 is disposed within the receiving cavity and extends into the slot 302 through the socket. The elastic pad 306 includes a first side and a second side opposite to one another. The first side defines a retaining groove that engages with the retaining plate, while the second side forms a boss that abuts the bottom of the slot 302. In this embodiment, the socket is located on the outer wall of the slot 302 for receiving the elastic pad 306. The receiving cavity within the base 300 serves as the main housing for the elastic pad 306 and communicates with the external slot 302 via the socket. The retaining plate is positioned on the side of the receiving cavity opposite the socket and functions to limit the position of the elastic pad 306, preventing displacement. The retaining groove on the first side of the elastic pad 306 mates with the retaining plate to fix the elastic pad 306 securely within the receiving cavity, thereby avoiding shifting or falling out. The boss on the second side abuts the bottom of the slot 302 to further ensure positional stability and prevent the elastic pad 306 from slipping out of the slot 302.

This embodiment of the utility model allows the elastic pad 306 to be inserted into and removed from the socket, enabling convenient replacement of a worn elastic pad 306 without requiring replacement of the entire base 300. Such a design enhances maintainability and extends the overall service life of the device.

In an embodiment, the slot 302 of the base 300 is configured as an arc-shaped slot, and the elastic pad 306 is positioned at the middle of its length. Correspondingly, the second phototherapy portion of the phototherapy mask 200 is formed in an arc shape. This configuration further improves the stability of the phototherapy mask 200 during installation.

In an embodiment, the outer wall of the slot 302 of the base 300 is crescent-shaped. Along the height of the slot 302, the width gradually increases from bottom to top. Along the length of the slot 302, the width at both ends is smaller than the width at the middle, such that the slot 302 is slightly wider in the center and narrower at the ends. This structure facilitates smooth insertion of the second phototherapy portion of the phototherapy mask 200 into the base 300, while simultaneously providing secure retention. Accordingly, the stability of the phototherapy mask 200 when mounted on the base 300 is enhanced.

In an embodiment, the housing of the controller 100 comprises a handle and a third phototherapy unit. The handle is configured to be inserted into the socket 304 of the base 300. The third phototherapy unit is connected to the handle and houses a control panel and a phototherapy assembly. The handle facilitates convenient use of the controller 100 by allowing it to be comfortably held in the hand, thereby improving operational usability. Furthermore, when the controller 100 is not in use, the lower end of the handle can be stably inserted into the socket 304 of the base 300, ensuring safe storage and reducing the risk of misplacement. The phototherapy assembly provided within the third phototherapy unit enables localized body phototherapy, thereby broadening the functionality of the overall device.

In an embodiment, the housing of the controller 100 is further provided with a socket. The controller 100 comprises a rechargeable battery located within the handle of the housing. The rechargeable battery is electrically connected to the control board. The control board is further provided with a port, which corresponds to the socket. The port may be used both for charging the rechargeable battery and for electrically connecting to the wire of the phototherapy mask 200. In one example, the port may be a Type-C interface.

By utilizing a single port for both charging and external connection to the phototherapy mask 200, the product structure is simplified, the number of external interfaces is reduced, and user convenience is improved. The control board manages the charging process and also governs the independent or combined operation of the third light board and the phototherapy mask 200. For example, the phototherapy mask 200 may be used for facial treatment, while the third light board may be used for localized body treatment (e.g., hands, legs), thereby meeting the phototherapy needs of different body areas.

Specifically, when the device is to be charged, the user connects a Type-C charging cable to the port through the socket on the housing. When facial phototherapy is desired, the phototherapy mask 200 is connected through the port, and a treatment mode is selected via the control panel, with the third light board deactivated. When both facial and localized body therapy are desired, the phototherapy mask 200 may be connected and the third light board activated simultaneously, with independent mode selection available via the control panel. Conversely, for body phototherapy alone, the phototherapy mask 200 may be disconnected, and the third light board can be operated independently.

For example, the phototherapy mask 200 may provide nine light therapy modes: red light plus near-infrared light, red light, blue light, green light, near-infrared light, violet light, yellow light, dark cyan light, and white light. The third light board may provide five modes: red light, blue light, green light, near-infrared light, and violet light. It is understood that the available light therapy modes may be identical or different depending on design requirements.

Referring to FIG. 7, the base 300 not only serves as a storage and support structure for the phototherapy mask 200 and the controller 100, but also functions as a charging station for both components. The base 300 is internally equipped with a charging module comprising a plurality of charging pins and corresponding electrical contacts. The charging pins are disposed within the slot 302 and the socket 304 of the base 300, positioned such that when the phototherapy mask 200 and the controller 100 are respectively placed into the slot 302 and the socket 304, the charging pins automatically engage with the corresponding charging terminals or electrodes provided on the phototherapy mask 200 and the controller 100.

The engagement between the charging pins and the electrodes establishes an electrical connection that allows power to be supplied from the base 300 to the internal rechargeable batteries of the phototherapy mask 200 and the controller 100. The base 300 may be connected to an external power source through a charging interface, such as a Type-C or magnetic connector, which provides the required input power for the charging operation. The charging module within the base 300 may further include charging control circuitry configured to regulate voltage, current, and charging time for each connected device independently or simultaneously.

In an embodiment, the phototherapy mask 200 comprises a rechargeable battery disposed within its internal structure. The rechargeable battery is electrically connected to the first phototherapy light board 202 and the second phototherapy light board 216 through an internal power circuit. The rechargeable battery enables the phototherapy mask 200 to operate independently without a continuous wired connection to the controller 100, thereby enhancing user convenience and portability during use. When the phototherapy mask 200 is placed in the slot 302 of the base 300, the charging pins 308 located at the bottom of the slot 302 automatically contact the charging electrodes of the rechargeable battery, thereby initiating charging.

Similarly, the controller 100 includes its own rechargeable battery 124, which may also be charged when the controller 100 is inserted into the socket 304 of the base 300. The charging pins disposed within the socket 304 engage with corresponding charging terminals on the housing 102 of the controller 100 to deliver charging current to the rechargeable battery 124. This automatic charging arrangement ensures that both the phototherapy mask 200 and the controller 100 are maintained in a fully charged state whenever they are docked in the base 300.

The provision of the charging pins and integrated charging circuitry within the base 300 enables simultaneous and efficient charging of multiple components without the need for separate charging cables. This design simplifies user operation, reduces clutter, and ensures continuous readiness of the system. Furthermore, the base 300 may include a control indicator or LED display configured to show the charging status of the phototherapy mask 200 and the controller 100, thereby providing visual confirmation of power connection and charging progress.

Through this configuration, the base 300 performs triple functions, serving as a storage station, a stable support platform, and a charging dock for the phototherapy mask 200 and the controller 100, thereby improving overall usability, operational convenience, and system integration.

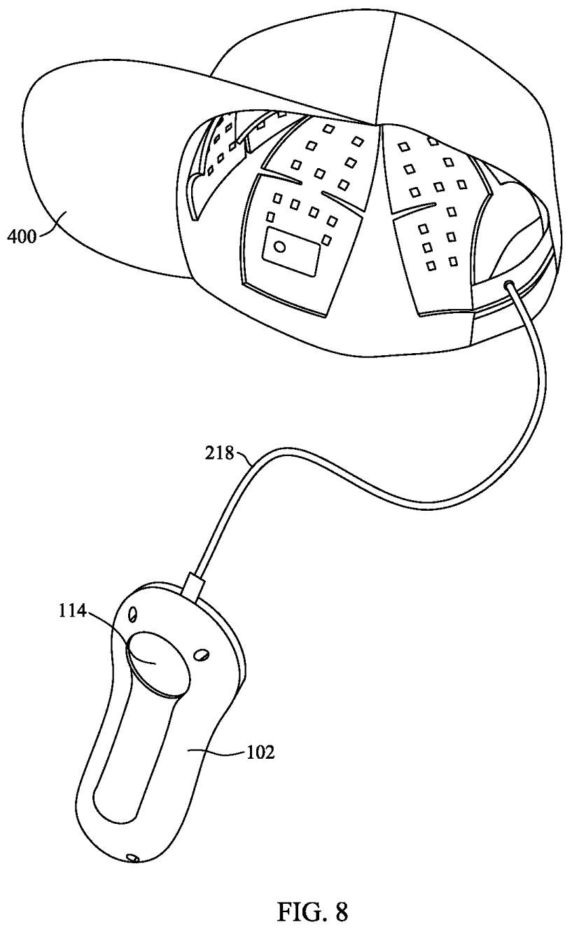

Referring to FIG. 8, in an embodiment, a phototherapy cap 400 is shown in connection with the controller 100. The phototherapy cap 400 represents an example of an alternative external therapeutic device that can be used interchangeably with the phototherapy mask 200 described in the previous embodiment. The phototherapy cap 400 is configured to conform to the contour of a user's head and to provide therapeutic light to the scalp, hair roots, and upper cranial regions for applications such as hair growth stimulation, scalp relaxation, and overall photobiomodulation therapy.

The phototherapy cap 400 comprises a phototherapy light board. The phototherapy light board is provided with a plurality of light-emitting elements, such as light-emitting diodes (LEDs) or micro-LEDs, disposed in a uniform array to ensure consistent light coverage across the scalp area. The inner surface is preferably formed of a flexible, thermally conductive, and light-transmitting material that enables comfortable contact with the user's scalp while allowing effective emission of therapeutic light. The outer surface provides mechanical support and protection for the internal components, ensuring durability and stability during operation.

In one embodiment, the phototherapy cap 400 is provided with a connector wire extending from the outer surface. The connector wire terminates in a coupling interface configured to engage with the port of the controller 100. The coupling interface may be magnetic, snap-fit, or a Type-C wired connection, enabling quick attachment and detachment between the phototherapy cap 400 and the controller 100. Once connected, the control board of the controller 100 is configured to regulate the operational parameters of the phototherapy cap 400, including activation, deactivation, wavelength selection, intensity level, and treatment duration.

The phototherapy cap 400 may be equipped with its own rechargeable battery disposed within the outer surface, electrically connected to the phototherapy light board. This configuration allows the cap to operate independently for a certain duration without being tethered to the controller 100. When the controller 100 and the phototherapy cap 400 are connected, power may be supplied directly from the controller 100 to the phototherapy cap 400, or the controller may communicate control signals while the cap operates on its internal power source. The dual power configuration increases operational flexibility and ensures uninterrupted therapy even when one power source is depleted.

In operation, the user may connect the phototherapy cap 400 to the controller 100 via the connector wire and select a desired therapy mode using the controller's user interface. The controller 100 delivers power and control signals to the phototherapy cap 400, thereby activating the LEDs on the third phototherapy light board to emit light of predetermined wavelengths. The emitted light penetrates the scalp tissue, stimulating cellular activity, enhancing blood circulation, and promoting follicle vitality. The intensity, duration, and wavelength of the emitted light can be adjusted via the controller's display or key module, ensuring personalized treatment according to user requirements.

In another embodiment, instead of the phototherapy cap 400, other forms of external phototherapy devices may be connected to the controller 100. These may include, but are not limited to, a phototherapy mask, a phototherapy pad, a phototherapy patch, a phototherapy band, or other wearable therapeutic modules designed for different anatomical regions such as the neck, shoulders, abdomen, arms, legs, or back. Each of these devices is configured with light-emitting elements and corresponding coupling structures to electrically and mechanically connect with the controller 100. The controller 100 may selectively operate one or more of these devices simultaneously or independently, thereby providing comprehensive and adaptable phototherapy coverage across multiple body areas.

Through this modular configuration, the system of the present invention achieves high versatility by enabling the controller 100 to function as a universal control and power interface for a range of phototherapy accessories. The user can interchangeably connect different therapeutic modules, such as a facial mask, scalp cap, flexible pad, or wearable patch, according to the targeted body area and required treatment type. This design significantly enhances usability, portability, and customization, allowing a single system to deliver multiple forms of light therapy across varied anatomical regions with precise control and optimal convenience.

In one example, the controller may include an automatic module recognition system configured to identify the type of connected therapy device based on electrical signature, resistance value, or data communication handshake. Upon detection, the controller automatically loads corresponding operational parameters or therapy modes optimized for that specific device, ensuring safe and efficient use of interchangeable therapy modules.

In an embodiment, the therapy system is configured to deliver therapies targeting both the central nervous system (CNS) and peripheral stimulation sites of the user. The first therapy device may be positioned or configured to stimulate a CNS-related site, while the therapeutic controller provides a second therapy to a peripheral region. The system is designed such that the first and second therapies are delivered simultaneously or in a coordinated manner to activate a neural pathway extending between the CNS stimulation site and the peripheral stimulation site.

In certain configurations, the delivery of the first and second therapies may be synchronized in frequency, phase, or timing, thereby enhancing neural activation and facilitating improved communication between central and peripheral regions of the nervous system. This coordinated operation may promote neural recovery, relaxation, or targeted stimulation for therapeutic and cosmetic applications.

In certain embodiments, synchronization between the first and second therapies may be configured to achieve neural entrainment, wherein rhythmic stimulation frequencies applied to central and peripheral regions align to enhance neural communication pathways. This may facilitate improved relaxation, recovery, or neuromodulation effects by coordinating photonic and electrical stimulation patterns.

Furthermore, the therapeutic controller may be configured to alternately or sequentially deliver the first and second therapies according to a coordinated timing sequence. For instance, when the first therapy device is delivering phototherapy to the facial area, the controller may temporarily suspend or reduce output from the second stimulation element, resuming operation once the facial session is complete. This controlled alternation allows the system to optimize energy use while maintaining balanced and effective treatment results.

Referring to FIGS. 5 and 8, in an embodiment, a method for providing one or more therapies to a user is provided, using a multifunctional therapy system comprising a first therapy device (a phototherapy mask 200) and a controller 100 configured to operate cooperatively. The phototherapy mask 200 and the controller 100 are designed to communicate with each other through a wired or wireless connection, thereby enabling synchronized or independent operation for providing multi-regional therapeutic treatments.

In certain embodiments, the therapeutic controller may include an artificial intelligence (AI) or machine-learning module configured to analyze stored therapy data, physiological sensor inputs, and user responses. Based on these analyses, the AI module can automatically recommend, predict, or adjust therapy parameters such as duration, wavelength, or stimulation pattern for optimized performance. The AI module may also learn user-specific preferences and create adaptive therapy programs for personalized treatment outcomes.

In certain embodiments, the system includes a wireless communication interface, such as Bluetooth, Wi-Fi, or cellular connectivity, enabling communication between the therapeutic controller and an external computing device or cloud server. A mobile application or web platform may be used to control therapy parameters, monitor session data, receive AI-driven recommendations, or share therapy progress with healthcare professionals. The wireless module enables firmware updates, remote diagnostics, and cloud-based data analytics for continuous improvement and remote management.

The therapeutic controller may further include internal or cloud-based storage configured to maintain a user profile comprising treatment history, preferred modes, physiological data, and AI-adjusted therapy settings. The stored data enables progress tracking and adaptive therapy recommendations over time.

In various embodiments, the first therapy device may employ a microcurrent electrode assembly as the primary stimulation element configured to deliver low-level electrical current to a first treatment region of the user's body. The microcurrent electrode may be arranged on a flexible pad, handheld applicator, or wearable module, and is configured to promote localized tissue stimulation, improve microcirculation, and activate neuromuscular pathways. The amplitude and frequency of the microcurrent may be adjusted by the therapeutic controller according to the selected therapy mode or based on user feedback.

In certain embodiments, the therapeutic controller includes one or more secondary stimulation elements, such as a phototherapy light source, vibration element, or pulsed electromagnetic field (PEMF) coil, which are adapted to deliver a second therapy to a different region of the user's body. For example, while the first therapy device provides microcurrent stimulation to a localized muscle or facial region, the therapeutic controller may simultaneously emit phototherapeutic light or PEMF energy to another area, such as the neck, shoulder, or hand region. This configuration enables multi-site and multimodal therapy, improving systemic relaxation and neurophysiological coordination.

In another embodiment, the first and second stimulation elements may utilize different therapeutic modalities to target complementary physiological effects. For instance, the first therapy device may use pulsed electromagnetic field (PEMF) stimulation to enhance cellular metabolism in a target tissue, while the second stimulation element in the controller applies microcurrent or EMS stimulation to facilitate muscle activation. Alternatively, the first therapy device may employ a phototherapy module with red, blue, or near-infrared light sources for dermatological benefits, while the second stimulation element in the controller provides thermal therapy through a heating or Peltier element to assist in collagen stimulation or relaxation.

It will be appreciated that numerous combinations of stimulation types and therapy regions are possible within the scope of the present invention. For example, combinations of microcurrent and PEMF, phototherapy and vibration, EMS and thermal therapy, or cooling and phototherapy may be employed. Each combination can be selected based on the therapeutic objective, such as pain relief, muscle recovery, skin rejuvenation, or relaxation. The modular design of the first therapy device and therapeutic controller allows the system to be customized or reconfigured to suit individual user needs and specific application areas.

The system may further be configured such that the first and second therapies are delivered concurrently or in a coordinated sequence, depending on the desired treatment protocol. In one implementation, simultaneous stimulation from both the first and second devices may be used to activate central and peripheral neural pathways in a synchronized manner. In another implementation, the controller alternates delivery between the two stimulation sources to prevent overstimulation and to achieve dynamic, phase-shifted activation of different body regions.

In certain embodiments, the therapy system is designed to deliver coordinated stimulation to both central nervous regions and peripheral stimulation sites of the user to facilitate neural pathway activation and communication. As used herein, the term central nervous region refers to body areas associated with or proximal to the spinal cord, cervical, or cranial nerve regions, such as the base of the neck, upper back, or scalp. The term peripheral stimulation site refers to distal body areas innervated by peripheral nerves, such as the limbs, facial regions, or extremities.

In one embodiment, the first therapy device may be positioned to stimulate a central nervous region, for example, a PEMF or microcurrent applicator placed near the cervical spine or occipital area to modulate neuronal excitability. Concurrently, the therapeutic controller, held or applied by the user at a peripheral region such as the wrist, forearm, or facial area, delivers a secondary stimulation such as vibration, phototherapy, or EMS. The combined activation establishes a communication loop between the stimulated central region and the peripheral site, enhancing signal propagation along afferent and efferent neural pathways.

In another embodiment, the first therapy device applies electromagnetic or photonic stimulation to a cranial or upper spinal region, while the second therapy device incorporated within the controller delivers microcurrent or EMS pulses to a distal muscle group. The system coordinates timing, frequency, or pulse phase between these two stimulation sources to synchronize neural firing patterns and promote neuromuscular facilitation. Such synchronized activation can improve muscle control, relaxation, or pain modulation through entrainment of central and peripheral neuronal circuits.

The therapeutic controller may include a synchronization module that manages timing relationships between the central and peripheral stimuli. In some implementations, the central region stimulation is delivered slightly in advance of the peripheral signal to prime neuronal responsiveness; in others, both are triggered simultaneously to reinforce bidirectional neural transmission. The coordinated delivery may follow preprogrammed protocols or adapt dynamically based on sensor feedback, thereby forming a closed-loop neurostimulation system.

This collaborative operation between the first therapy device and the therapeutic controller enables comprehensive neuro-physiological modulation, extending the therapeutic effect beyond the local application site. By concurrently engaging central and peripheral components of the nervous system, the system can achieve outcomes such as improved circulation, reduced muscle tension, enhanced relaxation, or recovery of neuromuscular coordination. The principle is applicable across a range of modalities, including combinations of PEMF with EMS, microcurrent with phototherapy, or heating with vibration therapies, depending on the target pathway and intended benefit.

The method enables a coordinated, dual-device therapeutic operation in which the first therapy device and the therapeutic controller communicate via wired or wireless connection. When connected, the handheld controller functions as a control center for the first therapy device, managing and synchronizing the delivery of multiple therapy types. Both devices can operate simultaneously or sequentially to provide therapy to different regions of the body, enhancing treatment versatility, efficiency, and user convenience. This integrated operational method allows the user to achieve multi-regional therapeutic effects through a single intelligent system designed for comprehensive wellness and rehabilitation.

In an embodiment, the method comprises the step of delivering a first therapy to a first treatment region of the user through the first therapy device. The first therapy device includes one or more first stimulation elements configured to provide specific forms of stimulation, such as phototherapy, microcurrent stimulation, thermal therapy, or vibrational therapy. The first therapy device may be shaped or structured as a mask, pad, cap, or wearable unit adapted to conform to a selected body region, such as the face, neck, scalp, or torso. The first stimulation elements, which may include LED lamp beads, heating plates, or electrode pads, are controlled by electrical signals transmitted from the therapeutic controller or from the internal control circuitry when operating independently.

The method further includes transmitting a control signal from the therapeutic controller to the first therapy device. The therapeutic controller includes a housing accommodating a control board, a power supply, and one or more second stimulation elements configured to deliver a second therapy to another treatment region of the body. The controller is equipped with a user input interface, such as a touch-sensitive display, which allows the user to select operational parameters, including therapy mode, wavelength, temperature, duration, or intensity. Once the user selects the desired parameters, the controller transmits a corresponding control signal to the first therapy device via a wired connection, such as a magnetic charging cable or dedicated connector, or through a wireless communication protocol, such as Bluetooth or near-field communication (NFC).

When the first therapy device and the therapeutic controller are connected, the controller automatically functions as a master control unit, coordinating and regulating the operation of both devices. The control signal from the controller enables real-time synchronization and adjustment of therapy output from the first therapy device. The system is configured such that one device, typically the handheld therapeutic controller, acts as a central control hub for the other connected device, managing power distribution, timing, and therapy parameters for both units. This intelligent communication allows the system to operate seamlessly, whether in a linked dual-device mode or as independent standalone units.

During operation, the method includes delivering a second therapy from the therapeutic controller to a different region of the user's body, such as the hands, arms, or legs, while the first therapy device continues to deliver the first therapy to the face, neck, or scalp. The system is capable of simultaneous dual-region therapy, enabling the user to receive treatments at multiple body sites concurrently. For example, while the first therapy device provides facial phototherapy, the therapeutic controller may provide localized microcurrent or heating therapy to the hands or arms. This simultaneous operation improves efficiency and convenience, allowing users to perform comprehensive treatments within a shorter duration.

In one configuration, the system may also operate in a coordinated sequential mode, wherein the first and second therapies alternate in a predetermined sequence. This coordinated sequence may be controlled by the timing logic on the control board to ensure that energy output is balanced and optimized for safety and efficacy.

The method further comprises the step of monitoring physiological parameters of the user during therapy through one or more sensors integrated into the therapeutic controller. The sensors may include a temperature sensor, impedance sensor, motion sensor, heart rate sensor, skin measuring sensor, or skin conductivity sensor, which continuously collect real-time data from the user's skin or tissue. Based on the detected parameters, the therapeutic controller may automatically adjust therapy parameters such as light intensity, current amplitude, or temperature level to maintain optimal treatment conditions. This feedback-based control ensures that therapy is delivered safely and effectively, personalized to the user's physiological state.

The therapeutic system may operate in a closed-loop feedback mode, wherein sensor outputs are continuously analyzed in real time to dynamically modulate therapy parameters. For example, if a temperature sensor detects elevated skin temperature, the controller automatically reduces light intensity or current amplitude to maintain safety thresholds. Conversely, if low skin impedance or reduced circulation is detected, stimulation intensity may be increased within safe limits to sustain therapeutic effectiveness.

In another example, the system may communicate with external wearable sensors, such as smartwatches or biometric patches, to receive additional physiological parameters (e.g., heart rate, oxygen saturation). This multi-source data integration allows the controller to adjust therapy intensity and duration based on overall user health metrics.

In one embodiment, the therapeutic controller further comprises one or more physiological sensors integrated into or mounted on its housing to monitor real-time physiological parameters of the user during therapy. The sensors may include, but are not limited to, a temperature sensor (e.g., thermistor or infrared sensor) configured to detect the surface temperature of the user's skin, skin measuring sensor, an electromyography (EMG) sensor configured to detect muscle activity, and a photoplethysmography (PPG) sensor or optical heart rate sensor configured to detect heart rate variability (HRV). These sensors are electrically coupled to the control board within the controller and continuously transmit physiological data during therapy sessions.

The control board of the therapeutic controller comprises a feedback processing module configured to receive the detected physiological data and dynamically adjust delivery of the second therapy based on the user's condition. For example, when the detected skin temperature exceeds a predefined threshold, the controller may automatically reduce the thermal output or intensity of the second therapy. Similarly, when muscle activity detected by the EMG sensor indicates excessive contraction, the controller may decrease the amplitude or duty cycle of microcurrent or EMS stimulation. In another example, variations in heart rate variability may trigger modulation of light frequency or pulse rate to maintain optimal comfort and safety.

Through this closed-loop feedback mechanism, the therapeutic controller achieves adaptive control, maintaining therapy output within a safe and effective range in real time. The feedback system allows personalization of therapy according to individual physiological responses, improving therapeutic efficacy while reducing the risk of overheating, overstimulation, or user discomfort.

The system may also include adaptive synchronization functionality, wherein the first and second therapies are synchronized in terms of frequency, phase, or duration. Such synchronization may be advantageous when both devices are targeting regions connected through a neuromuscular or neural pathway, enabling simultaneous activation of central and peripheral areas. For instance, when the first therapy device provides stimulation to the scalp (central nervous region) and the therapeutic controller provides stimulation to the hand or arm (peripheral region), synchronized operation enhances neural responsiveness and therapeutic outcomes.

Additionally, the method may include wireless data transmission between the devices for parameter adjustment, data logging, and user progress tracking. The controller can store therapy records, usage time, and sensor data in memory, allowing the system to recommend optimized therapy settings in future sessions based on previous performance and physiological feedback.

The present invention provides an advanced dual-function therapy system integrating a first therapy device and a therapeutic controller capable of delivering multiple therapeutic modalities either independently or in a coordinated manner. By incorporating stimulation elements such as phototherapy emitters, microcurrent electrodes, vibration actuators, and thermal regulation components, the system enables comprehensive and customizable treatment across different regions of the body. The ability of the devices to communicate through wired or wireless connections allows seamless synchronization and adaptive control based on user-selected parameters or real-time physiological feedback. This versatile configuration supports a wide range of medical, cosmetic, and wellness applications, including skin rejuvenation, muscle relaxation, neural stimulation, pain management, and circulation enhancement.

The invention is particularly suitable for industrial applications in the fields of medical aesthetics, rehabilitation technology, wellness electronics, and wearable healthcare devices. Its modular and scalable design facilitates integration into consumer-grade and professional therapeutic equipment, enabling efficient manufacturing, portability, and user adaptability. Through the combination of intelligent control, multi-modal therapy, and ergonomic design, the invention offers a significant advancement in next-generation personal therapy systems, bridging the gap between clinical treatment precision and everyday usability.

The system may further find application in cosmetic skincare treatments such as anti-aging, acne reduction, pigmentation correction, and collagen regeneration, as well as wellness domains including stress relief, sleep enhancement, and circulation improvement. Through intelligent modulation of light and electromagnetic stimulation, the invention bridges medical-grade precision and consumer-level convenience.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A therapy system, comprising:
a first therapy device having one or more first stimulation elements configured to deliver a first therapy to a first treatment region of a user;
a therapeutic controller in communication with the first therapy device, the therapeutic controller comprising:
a user input interface configured to generate a control signal for operation of the first therapy device, and
one or more second stimulation elements configured to deliver a second therapy to a second treatment region of the user; and
wherein the second therapy is delivered independently or concurrently with the first therapy.

2. The therapy system of claim 1, wherein the one or more first stimulation element and the one or more second stimulation element is selected form a group consisting of a phototherapy element, a microcurrent element, a pulsed electromagnetic field (PEMF) generator, an electromagnetic field (EMF) generator, an EMS element, a heating element, a cooling element, a Peltier element, a vibrator element or a piezoelectric element.

3. The therapy system of claim 1, wherein the one or more first stimulation elements and the one or more second stimulation elements comprise a phototherapy unit including at least one light source having a plurality of lamp beads.

4. The therapy system of claim 3, further comprising a lens assembly including a light-transmitting plate and a condenser lens aligned with the plurality of lamp beads.

5. The therapy system of claim 1, wherein the first therapy device further comprises a port configured to enable charging of a battery and connection to an external device.

6. The therapy system of claim 5, wherein the port is configured to connect to an external phototherapy device through a wired connection.

7. The therapy system of claim 1, wherein the therapeutic controller comprises at least one physiological sensor selected from a temperature sensor, an impedance sensor, a motion sensor, a heart rate sensor, skin measuring sensor, or a skin conductivity sensor.

8. The therapy system of claim 6, wherein the therapeutic controller is configured to adaptively adjust delivery of the second therapy based on feedback from the at least one physiological sensor.

9. The therapy system of claim 1, wherein the user input interface comprises a touch-sensitive display configured to receive user input to select therapy modes, duration, or intensity.

10. A therapy system comprising:
a first therapy device having one or more first stimulation elements configured to deliver a first therapy to a central nervous region stimulation site of a subject;
a therapeutic controller in communication with the first therapy device, the therapeutic controller comprising a user input interface and at least one second stimulation element configured to deliver a second therapy to a peripheral stimulation site of the subject; and
wherein the first therapy and the second therapy are delivered simultaneously or in a coordinated manner to activate a neural pathway extending between the central nervous system stimulation site and the peripheral stimulation site.

11. The therapy system of claim 10, wherein the one or more first stimulation element and the one or more second stimulation element is selected form a group consisting of a phototherapy element, a microcurrent element, a pulsed electromagnetic field (PEMF) generator, an electromagnetic field (EMF) generator, an EMS element, a heating element, a cooling element, a Peltier element, a vibrator element or a piezoelectric element.

12. The therapy system of claim 10, wherein the first therapy and the second therapy are synchronized in frequency, phase, or timing to enhance neural pathway activation.

13. The therapy system of claim 10, wherein the therapeutic controller further comprises a physiological sensor configured to detect a parameter of the subject and adjust delivery of the second therapy based on the detected parameter.

14. The therapy system of claim 13, wherein the parameter comprises at least one of skin temperature, muscle activity, or heart rate variability.

15. The therapy system of claim 10, wherein the therapeutic controller is configured to operate the second stimulation element while transmitting a control signal to regulate delivery of the first therapy.

16. The therapy system of claim 10, wherein the therapeutic controller and the first therapy device are configured to alternate delivery of the first therapy and the second therapy in a coordinated sequence.

17. A method for providing one or more therapies to a user, the method comprising delivering a first therapy to a treatment region using a first therapy device having one or more first stimulation elements;
transmitting, from a therapeutic controller, a control signal to the first therapy device to regulate the first therapy; and
delivering, concurrently or sequentially, from the therapeutic controller, a second therapy to the user using at least one second stimulation element integrated into the therapeutic controller.

18. The method of claim 17, further comprising detecting a physiological parameter of the user using a sensor in the therapeutic controller.

19. The method of claim 18, further comprising adjusting at least one of the first therapy or the second therapy based on the detected physiological parameter.

20. The method of claim 17, wherein delivering the first therapy and delivering the second therapy are performed simultaneously in a synchronized manner.
